(19) United States
(12) Patent Application Publication
(10) Pub. No.: US 2012/0315437 A1
(43) Pub. Date: Dec. 13, 2012

(54) NOVEL MICROORGANISM CAPABLE OF PRODUCING OXIDE

(76) Inventors: Jun Takada, Okayama-shi (JP); Michinori Sawayama, Okayama-shi (JP); Tomoko Suzuki, Okayama-shi (JP); Hideki Hashimoto, Okayama-shi (JP); Tatsuo Fujii, Okayama-shi (JP); Makoto Nakanishi, Okayama-shi (JP)

(21) Appl. No.: 13/515,943

(22) PCT Filed: Dec. 14, 2010
(86) PCT No.: PCT/JP2010/072503

§ 371 Date: Jun. 14, 2012

(30) Foreign Application Priority Data
Dec. 15, 2009 (JP) 2009-284445
Jan. 8, 2010 (JP) 2010-003269

(51) Int. Cl. C12N 1/21; C01G 49/04; C12P 3/00; C01G 49/06; C12N 1/20; C12Q 1/02

(52) U.S. Cl. 428/141; 435/252.1; 435/252.3; 435/29; 435/168

(57) ABSTRACT

Disclosed is a microorganism that belongs to the genus Leptothrix and is capable of producing iron oxide that has a ferrihydrite or lepidocrocite structure and has a form of aggregates of ferrihydrite nanoparticles or lepidocrocite nanoparticles; a bacterium that is capable of producing an iron oxide that has a ferrihydrite or lepidocrocite structure and has a form of aggregates of ferrihydrite nanoparticles or lepidocrocite nanoparticles; a culture medium for use in screening a bacterium that is capable of producing a metal oxide; a method for screening a bacterium that is capable of producing a metal oxide; a culture medium for culturing a bacterium that is capable of producing a metal oxide; a method for culturing a bacterium that is capable of producing a metal oxide; a method for producing a metal oxide; and iron oxide.

### TECHNICAL FIELD

The present invention relates to a microorganism that belongs to the genus Leptothrix and is capable of producing iron oxide, a bacterium that is capable of producing iron oxide, a culture medium for use in screening a bacterium that is capable of producing a metal oxide, and a method for screening a bacterium that is capable of producing a metal oxide. The present invention further relates to a culture medium for culturing a bacterium that is capable of producing a metal oxide, and a method for culturing a bacterium that is capable of producing a metal oxide. The present invention also relates to a method for producing a metal oxide, and a novel metal oxide.

### BACKGROUND ART

Materials that have a unique shape, size, and composition may have innovative functions and are therefore important. In particular, materials of a unique shape, size, and composition that cannot be made artificially have enormous potential for applications. For example, it is known that microorganisms that belong to the genus Leptothrix inhabit swamps and springs that are rich in iron and manganese, and form a sheath-shaped substance comprising iron oxide and/or manganese oxide. A recent study revealed that these sheath-shaped substances derived from microorganisms are inorganic materials with an attractive structure of unique microtube, and that they are applicable to various industrial fields.

Microorganism-derived ceramic materials produced by this kind of iron bacteria, which clog pipes and cause red water, have simply been treated as waste. However, microorganism-derived ceramic materials are worthy of greater attention because they are derived from organisms and are thus environmentally friendly, and they mainly consist of the ubiquitous elements iron and silicon, and are thus a continuously available, unutilized resource. Moreover, any attempt to artificially produce such a unique structure would require a huge amount of time and effort as well as immense technology and energy. Accordingly, the development of novel materials by utilizing ceramic materials derived from nature is highly significant in terms of both of science and technology.

Patent Literature 1 discloses a method for collecting sheath-shaped iron oxide from aggregate generated in a water purifying process using iron bacteria. Specifically, Patent Literature 1 discloses a method for producing sheath-shaped iron oxide particles, wherein an aggregated sedimentation formed in a biological water purification method that uses ferrobacteria is reacted with a dispersant (Hydrangea paniculata extract or Abelmoschus Manihot extract). The pipe-shaped iron oxide collected by this method has a unique composition/shape and excellent properties; therefore, it is useable as a magnetic material, catalyst, adsorbent and battery material.

The method for collecting the sheath-shaped iron oxide described in Patent Literature 1 uses various iron bacteria that exist in nature to form an aggregate; therefore, it is difficult to completely remove substances other than the sheath-shaped iron oxide. Furthermore, the water supplied is natural water, so the temperature, ion content, etc., cannot be controlled; therefore, the yield thereof is unstable and the same composition cannot always be obtained. Furthermore, the sheath-shaped substance must be purified in order to use it as an industrial material.

The quickest way to solve these problems is to isolate the iron bacteria that are capable of forming sheath-shaped iron oxide, and find the culture conditions under which a sheath-shaped oxide can be formed, using this isolated bacteria.

Various methods using a low nutrient culture medium have been reported as a method for isolating iron-oxidizing bacteria selected from the group consisting of unculturable and aerobic iron-oxidizing bacteria, exemplified by the genus Leptothrix (Non-patent Literature 1). However, because these culture media contain organic matter, such as hydrocarbons, a variety of bacteria other than the target bacteria can proliferate. Therefore, it hardly serves as a selective culture medium for the target. In addition to the methods described above, a continuous culture system that simulates the natural habitat has also been devised (Non-patent Literature 2). The use of this method increases the probability of isolation; however, it has drawbacks, such as the fact that a large-scale system is required and it is impossible to obtain only one type of strain.

As described above, a method for isolating a microorganism that is capable of producing sheath-shaped iron oxide has not been established, and the properties of such a microorganism, as well as the oxidation mechanisms of iron and manganese, have not yet been revealed.

To date, the isolation of Leptothrix cholodnii SP-6 strain, which is a sheath-forming strain (Non-patent Literature 3 and 4), and the isolation of Leptothrix cholodnii SA-1 strain, which produces sheath-shaped iron oxide (Non-patent Literature 5 and 6) have been reported.

### CITATION LIST

### Patent Literature

PTL 1: Japanese Unexamined Patent Publication No. 2005-272251

### Non-Patent Literature

NPL 1: Spring, S. The genera Leptothrix and Sphaerotilus. Prokaryotes 5, 758-777 (2006)
NPL 2: Mulder, E. G., and W. L. van Veen, Investigations on the Sphaerotilus-Leptothrix group. Ant, v. Leeuwhoek 29, 121-153 (1963)
NPL 3: Emerson, D. and Ghiorse, W. C., Isolation, Cultural Maintenance, and Taxonomy of a Sheath-Forming Strain of Leptothrix discophora and Characterization of Manganese-Oxidizing Activity Associated with the Sheath. Appi. Environ. Microbiol. 58, 4001-4010 (1992)
NPL 4: Spring, S., Kampfer, P., Ludwig, W. and Schleifer, K. H., Polyphasic characterization of the genus Leptothrix: new descriptions of Leptothrix mobilis sp. nov. and Leptothrix discophora sp. nov. nom. rev. and emended description of Leptothrix cholodnii emend Syst. Appl. Microbiol. 19, 634-643 (1996).
NPL 5: PROGRAM and ABSTRACT 6^{th} International Symposium on Electron Microscopy in Medicine and Biology 2009 (6^{th} ISEM09), Sep. 16, 2009, p. 50
NPL 6: Abstracts of the Meeting of the Society for Biotechnology, Japan, p. 125, 2Ia15, The Society for Biotechnology, Japan, Aug. 25, 2009

### SUMMARY OF INVENTION

### Technical Problem

However, the Leptothrix cholodnii SP-6 disclosed in Non-patent Literature 3 and 4 has the following drawbacks. It cannot metabolize many kinds of organic substances, it exhibits a weak adhesion to cells, it has a low sheath-producing capability, it is insufficiently able to maintain a sheath-producing capability, etc.

Furthermore, Non-patent Literature 5 and 6 do not disclose the properties of the Leptothrix cholodnii SA-1 strain, the isolation method thereof, the culturing method thereof, the production method of a sheath-shaped substance, etc.

The present invention aims to provide, by isolating novel iron bacteria from nature, which has been conventionally difficult, a microorganism that is capable of producing a specific iron oxide and that belongs to the genus Leptothrix, a bacterium that is capable of producing a specific iron oxide, a culture medium for use in screening a bacterium that is capable of producing a metal oxide, and a method for screening a bacterium that is capable of producing a metal oxide. The present invention also aims to provide a culture medium for use in culturing a bacterium that is capable of producing a metal oxide, and a method for culturing a bacterium that is capable of producing a metal oxide. The present invention further aims to provide a method for producing a metal oxide, and a novel iron oxide.

### Solution to Problem

The present inventors found that, by using a specific culture medium, a novel iron bacterium that was conventionally difficult to isolate from nature, can be isolated from groundwater sediment contained in a storage tank at a water purification plant wherein tap water is obtained using ground water. The present inventors also found a culture medium that can facilitate the proliferation of the bacterium and the production of iron oxide at the same time.

The present invention has been accomplished based on these findings. The present invention provides a microorganism that belongs to the genus Leptothrix and is capable of producing iron oxide, a bacterium that is capable of producing iron oxide, a culture medium for use in screening a bacterium that is capable of producing a metal oxide, a method for screening a bacterium that is capable of producing a metal oxide, and the like.

Item 1. A microorganism that belongs to the genus Leptothrix,

the microorganism being capable of producing an iron oxide having a structure of ferrihydrite or lepidocrocite, the iron oxide being an aggregate of ferrihydrite nanoparticles or lepidocrocite nanoparticles.

Item 2. The microorganism according to Item 1, wherein the iron oxide comprises phosphorus and silicon.

Item 3. The microorganism according to Item 1 or 2, which comprises 16S rDNA consisting of the nucleotide sequence of SEQ ID NO: 1.

Item 4. The microorganism according to any one of Items 1 to 3, which is Leptothrix cholodnii OUMS1 (NITE BP-860).

Item 5. A bacterium that is capable of producing an iron oxide having a structure of ferrihydrite or lepidocrocite, the iron oxide being an aggregate of ferrihydrite nanoparticles or lepidocrocite nanoparticles.

Item 6. The bacterium that is capable of producing an iron oxide according to Item 5, wherein the iron oxide contains phosphorus and silicon.

Item 7. A culture medium for use in screening a bacterium that is capable of producing a metal oxide, the culture medium comprising an inorganic phosphorus compound and an iron compound added to natural ground water.

Item 8. A method for screening a bacterium that is capable of producing a metal oxide comprising culturing the bacterium using the culture medium of Item 7.

Item 9. A culture medium for culturing a bacterium that is capable of producing a metal oxide, the culture medium comprising as medium components a carbon source, a nitrogen source, silicon, sodium, calcium, magnesium, potassium, inorganic phosphate, and iron.

Item 10. A method for culturing a bacterium that is capable of producing a metal oxide, the method comprising a step of using the culture medium of Item 9.

Item 11. A method for producing a metal oxide comprising:

culturing the microorganism of any one of Items 1 to 4 or the bacterium that is capable of producing an iron oxide of Item 5 or 6; and

collecting the metal oxide from a culture fluid.

Item 12. The method according to Item 11, wherein the metal oxide is in the shape of a microtube, a nanotube, a hollow string, a capsule, a string-and-sphere agglomerate, a string, or a rod.

Item 13. An iron oxide having a ferrihydrite or lepidocrocite structure, being an aggregate of ferrihydrite nanoparticles or lepidocrocite nanoparticles, and having a fibrous or scaly surface.

### Advantageous Effects of Invention

The present invention provides a method for screening novel iron bacteria, which was conventionally difficult to isolate from nature. The use of the microorganism, which belongs to the genus Leptothrix, of the present invention makes it possible to produce highly pure iron oxide. By using the microorganism belonging to the genus Leptothrix or a bacterium that is capable of producing iron oxide of the present invention, an iron oxide that is an aggregate of ferrihydrite nanoparticles or lepidocrocite nanoparticles can be produced.

Because it requires the culturing of only one kind of strain, the preservation/maintenance and control of culturing can be easily performed, and a stable yield of the metal oxide can be attained. Furthermore, by changing the composition of the culture medium or adding other materials, a metal oxide having a shape, composition and properties that do not exist in nature can be produced.

### BRIEF DESCRIPTION OF DRAWINGS

FIG. 1 shows an optical microscope image (A) and a scanning electron microscope (SEM) image (B) of the oxide in the shape of a sheath obtained after culturing an OUMS1 strain in a GP liquid medium.

FIG. 2-A shows the results of a homology search showing the 16S ribosomal DNA nucleotide sequence of an OUMS1 strain (upper row) and that of a known iron-oxidizing bacteria Leptothrix cholodnii SP-6 strain (lower row).

FIG. 2-B shows the results of a homology search showing the 16S ribosomal DNA nucleotide sequence of an OUMS1 strain (upper row) and that of a known iron-oxidizing bacteria Leptothrix cholodnii SP-6 strain (lower row).

FIG. 3 compares genomic DNA electrophoretic patterns of an OUMS1 strain (A) and an iron-oxidizing bacteria Leptothrix cholodnii SP-6 strain (B).

FIG. 4 shows an optical microscope image (A) and a SEM image (B) of the oxide in the shape of a sheath obtained after culturing an OUMS1 strain in a SIGP liquid medium.

FIG. 5-A shows SEM images of the iron oxide formed by an OUMS1 strain.

FIG. 5-B shows SEM images of the iron oxide formed by an OUMS1 strain.

FIG. 6 shows TEM images of the iron oxide formed by an OUMS1 strain.

FIG. 7 shows an X-ray diffraction (XRD) pattern of the iron oxide formed by an OUMS1 strain.

FIG. 8 shows a high-resolution TEM image of the iron oxide formed by an OUMS1 strain.

FIG. 9 shows an optical microscope image of the iron oxide in the shape of a sheath obtained after culturing an OUMS1 strain in a SIGP liquid medium.

FIG. 10 shows an X-ray diffraction (XRD) pattern of the iron oxide formed by an OUMS1 strain.

### DESCRIPTION OF EMBODIMENTS

The present invention is explained in detail below.

### Bacterium Capable of Producing Iron Oxide

The present invention provides a bacterium that is capable of producing an iron oxide that has a ferrihydrite or lepidocrocite structure, i.e., a low crystalline iron oxide, and that is an aggregate of ferrihydrite nanoparticles or lepidocrocite nanoparticles.

Ferrihydrite as used herein refers to a low-crystalline iron oxide. Ferrihydrite is called 2-line ferrihydrite, 6-line ferrihydrite, etc., depending on the number of peaks that appear in X-ray diffraction patterns thereof. The composition of 2-line ferrihydrite is Fe₄(O, OH, H₂O), and the composition of 6-line ferrihydrite is Fe_{4.6}(O, OH, H₂O)₁₂ (R. A. Eggleton and R. W. Fitzpatrick, “New data and a revised structural model for ferrihydrite,” Clays and Clay Minerals, Vol. 36, No. 2, pages 111 to 124, 1988).

Lepidocrocite is a crystalline iron oxide represented by the chemical formula of γ-FeOOH and having the following properties. Crystal system: orthorhombic system, space group: Bb mm, lattice constant: a=0.3071, b=1.2520, c=0.3873 Å, and α=β=γ=90°.

The iron oxide produced by iron oxide-producing bacteria may contain phosphorus and silicon. The primary particle diameter of ferrihydrite nanoparticles is preferably about 3 to 5 nm, and the primary particle diameter of lepidocrocite nanoparticles is preferably about 30 to 50 nm.

Although any bacterium that is capable of producing iron oxide having a ferrihydrite or lepidocrocite structure (a structure similar to that of ferrihydrite or lepidocrocite) may be used, the microorganism preferably belongs to the genus Leptothrix, and more preferably Leptothrix cholodnii. One example of such a microorganism is a Leptothrix cholodnii OUMS1 strain isolated from a water purification plant. The Leptothrix cholodnii OUMS1 strain can produce iron oxide having a ferrihydrite or lepidocrocite structure. Mycological and genetic properties of the Leptothrix cholodnii OUMS1 strain are shown below.

### (i) Mycological Properties

The Leptothrix cholodnii OUMS1 strain is a bacillus with a length of several micrometers and a width of about 1 micrometer. At the single-cell stage, this strain actively moves using a flagellum. As the cell grows, both ends of the cell are connected, and a fibrous material comprising a polysaccharide and a protein is formed around the cell. As a result, this cell cannot be uniformly present in a liquid medium and is in an aggregated and precipitated state. When iron and manganese are added to the medium, iron oxide and manganese oxide adhere to the fibrous material that is present outside of the cell, thus forming a sheath-shaped structure. The cell forms a white amorphous fibrous colony on an agar medium. When iron is added, the colony becomes yellowish brown. When manganese is added, the colony becomes brown.

### (ii) Genetic Properties

The nucleotide sequence of the 16S rDNA of the Leptothrix cholodnii OUMS1 strain is shown in SEQ ID NO: 1 of the Sequence Listing. A BLAST search was performed on the DDBJ database for the nucleotide sequence of 16S rDNA. The results of this search and the mycological properties described above confirmed that this cell belongs to Leptothrix cholodnii.

The Leptothrix cholodnii OUMS1 strain has the following properties. It can metabolize many kinds of organic substances, it exhibits strong adhesion to cells, it has a high sheath-producing ability and capability of maintaining the sheath-producing ability, etc. As a result, it enables the following effects to be achieved. Inexpensive organic substances can be selected, many cells adhere to its iron fragment so they can be involved in the production of iron oxide, iron oxide can be stably produced, etc.

The Leptothrix cholodnii OUMS1 strain was deposited as Accession No. NITE P-860 in the National Institute of Technology and Evaluation, Patent Microorganisms Depositary (Kazusa Kamatari 2-5-8, Kisarazu, Chiba, 292-0818, Japan) on Dec. 25, 2009. This bacterial strain has been transferred to an international deposit under Accession No. NITE BP-860.

In addition to the Leptothrix cholodnii OUMS1 strain, other examples of microorganisms that belong to the genus Leptothrix that are capable of producing iron oxide having a ferrihydrite or lepidocrocite structure include microorganisms that belong to the genus Leptothrix having 16S rDNA consisting of the nucleotide sequence shown in SEQ ID NO: 1. Specific examples of bacteria that are capable of producing iron oxide having a ferrihydrite or lepidocrocite structure include bacteria having 16S rDNA consisting of the nucleotide sequence shown in SEQ ID NO: 1.

### Culture Medium for Use in Screening Bacterium Capable of Producing Metal Oxide

The culture medium for use in screening a bacterium that is capable of producing a metal oxide of the present invention is characterized in that it comprises an inorganic phosphorus compound and iron compound added to natural ground water.

By the use of such a medium that does not require a carbon source, and that comprises iron and phosphorus, which are constituents of a metal oxide, added thereto, a bacterium that is capable of producing a metal oxide will preferentially proliferate.

Examples of the metal oxides include iron oxide (e.g., an iron oxide having a ferrihydrite or lepidocrocite structure), manganese oxide, and the like. The metals used here contain silicon and phosphorus. The metal oxide may be in the shape of a microtube, a nanotube, a hollow string, a capsule, a string-and-sphere agglomerate, a string, a rod, or the like.

The natural ground water is not limited as long as it was extracted from underground regardless of the place from which it was collected. However, the natural ground water preferably contains, respectively calculated as atoms, about 10 to 50 ppm and particularly preferably about 15 to 25 ppm of silicon, about 5 to 50 ppm and particularly preferably about 10 to 15 ppm of calcium, about 1 to 100 ppm and particularly preferably about 5 to 10 ppm of sodium, about 1 to 15 ppm and particularly preferably about 3 to 5 ppm of magnesium, and about 0.1 to 10 ppm and particularly preferably about 1 to 2 ppm of potassium. These elements usually exist in the medium in the form of silicate ions, calcium ions, sodium ions, magnesium ions, and potassium ions.

The unit ppm used in the present specification indicates an ionic concentration (mg/L).

The concentration of inorganic phosphate in the medium is preferably 1 to 50 ppm and particularly preferably 5 to 20 ppm, and the concentration of iron is preferably 0.01 to 1 mM and particularly preferably 0.03 to 0.1 mM. Examples of the inorganic phosphorus compounds include phosphate, polyphosphoric acid, pyrophosphoric acid, and the like. Examples of the iron compounds include ferrous sulfate, iron nitrate, an iron fragment, and the like.

The pH of the culture medium of the present invention that is used for screening is preferably in the neutral region and particularly preferably 7. The culture medium of the present invention used for screening may contain HEPES (i.e., 2-[4-(2-hydroxyethyl)-1-piperazinyl]ethanesulfonic acid) and like buffers.

One such example is a medium that comprises, as a basic component, natural ground water (e.g., natural ground water containing about 15 to 25 ppm of silicon, about 10 to 15 ppm of calcium, about 5 to 10 ppm of sodium, about 3 to 5 ppm of magnesium, and about 1 to 2 ppm of potassium), and, as additional components, about 10 ppm of inorganic phosphate ions, about 2.3 to 2.4 g of HEPES per liter of medium, about 0.01 to 0.05 mM of iron (II) sulfate and an iron fragment (purity: 99.9%, about 5 nm square) for which the pH is adjusted to 7.0. A more specific example thereof is a GP medium (containing 0.076 g of disodium hydrogenphosphate dodecahydrate, 0.02 g of potassium dihydrogenphosphate dihydrate, 2.383 g of HEPES, and 0.01 mM of iron sulfate, per liter of sterile groundwater, whose pH was adjusted to 7.0 with an aqueous sodium hydroxide solution).

The method for screening a bacterium that is capable of producing a metal oxide of the present invention is characterized in that the culturing is performed using the medium described above. By culturing with such a medium, it becomes possible to screen a bacterium that is capable of producing a metal oxide, and which was conventionally difficult to isolate from nature.

The culture form may be either liquid or solid and culturing may be performed in accordance with an ordinary method for culturing microorganisms. The culture conditions may be suitably selected depending on the characteristics of the microorganism to be screened. An example of culture temperature is 15 to 30° C. and preferably 20 to 25° C. The culture time cannot be uniformly generalized, and may usually be about 4 to 35 days, and preferably about 7 to 21 days.

For example, after repeating liquid culturing several times using the medium, a diluent of the culture fluid is inserted dropwise into and cultured in an agar plate medium to form a single colony, and screening can thereby be performed.

### Culture Medium for Culturing Bacterium Capable of Producing Metal Oxide

The culture medium for culturing a bacterium that is capable of producing a metal oxide of the present invention is characterized in that it contains, as the medium components, a carbon source, a nitrogen source, silicon, sodium, calcium, magnesium, potassium, inorganic phosphate, and iron.

By adjusting the mineral composition of the culture medium to be similar to that of ground water, adding a carbon source and a nitrogen source thereto to facilitate proliferation, and adding iron and phosphorus, which are the constituents for metal oxide, the production of iron oxide and the proliferation of an iron oxide-producing bacterium can be achieved.

Examples of the metal oxides include those mentioned above.

Examples of the carbon sources contained in the medium include glucose, sucrose, fructose, maltose, glycerin, dextrin, oligosaccharide, starch, molasses, corn steep liquor, malt extract, organic acid, and the like. The concentration of the carbon source is preferably 0.01 to 10 g/L, and particularly preferably 0.1 to 2 g/L.

Examples of the nitrogen sources contained in the medium include corn steep liquor, yeast extract, various peptones, soybean flour, meat extract, wheat bran extract, casein, amino acid, urea, and the like. The concentration of the nitrogen source is preferably 0.01 to 10 g/L, and particularly preferably 0.1 to 2 g/L.

The concentrations of the mineral components contained in the medium are preferably similar to those of ground water. Specifically, the medium preferably contains, respectively calculated as atoms, about 10 to 50 ppm and particularly preferably about 15 to 25 ppm of silicon, about 5 to 50 ppm and particularly preferably about 10 to 15 ppm of calcium, about 1 to 100 ppm and particularly preferably about 5 to 10 ppm of sodium, about 1 to 15 ppm and particularly preferably about 3 to 5 ppm of magnesium, and about 0.1 to 10 ppm and particularly preferably about 1 to 2 ppm of potassium. These elements usually exist in the medium in the form of silicate ions, calcium ions, sodium ions, magnesium ions, and potassium ions.

The concentration of the inorganic phosphate in the medium is preferably 1 to 50 ppm and particularly preferably 5 to 20 ppm, and the concentration of iron is preferably 0.01 to 1 mM and particularly preferably 0.03 to 0.1 mM. The inorganic phosphate may be added to the medium in the form of phosphate, polyphosphoric acid, pyrophosphoric acid, and the like; and the iron may be added to the medium in the form of ferrous sulfate, iron nitrate, an iron fragment, and the like.

The medium preferably has a pH in the neutral region, and particularly preferably 7. The medium of the present invention used for screening may contain HEPES and like buffers.

One such example is a medium to which 0.01 to 10 g of glucose, 0.01 to 10 g of peptone, 0.1 to 1 g of sodium metasilicate nonahydrate, 0.02 to 0.1 g of calcium chloride dihydrate, 0.01 to 0.1 g of magnesium sulfate heptahydrate, 0.02 to 0.2 g of disodium hydrogenphosphate dodecahydrate, 0.01 to 0.05 g of potassium dihydrogenphosphate dihydrate, 1 to 4 g of HEPES, 0.01 to 0.05 mM of iron (II) sulfate, and an iron fragment (purity: 99.9%, about 5 mm square), per liter of sterile distilled water, were added, and the pH thereof was adjusted to 7.0 with an aqueous sodium hydroxide solution. A specific example is an SIGP liquid medium (containing 1 g of glucose, 1 g of peptone, 0.2 g of sodium metasilicate nonahydrate, 0.044 g of calcium chloride dihydrate, 0.041 g of magnesium sulfate heptahydrate, 0.076 g of disodium hydrogenphosphate dodecahydrate, 0.02 g of potassium dihydrogenphosphate dihydrate, 2.383 g of HEPES, 0.05 mM of ferrous sulfate, per liter of sterile distilled water, whose pH was adjusted to 7.0 with an aqueous sodium hydroxide solution).

The method for culturing a bacterium that is capable of producing a metal oxide of the present invention is characterized in that the medium described above is used. By the use of such a medium, the production of iron oxide and the culturing of an oxide-producing bacterium both become possible.

The culture form may be either liquid or solid and culturing may be performed in accordance with an ordinary method for culturing microorganisms. For example, it can be performed by shaking a liquid culture. The culture conditions may be suitably selected depending on the characteristics of the metal oxide-producing bacterium that is to be cultured. An example of the culture temperature is 15 to 30° C. and preferably 20 to 25° C. The culture time cannot be uniformly generalized, and may be usually for about 7 to 35 days, and preferably about 7 to 21 days.

### Method for Producing Metal Oxide

The method for producing a metal oxide of the present invention is characterized in that the aforesaid microorganism that belongs to the genus Leptothrix or the bacterium that is capable of producing iron oxide is cultured and then metal oxide is collected from the culture fluid.

Examples of the metal oxides include iron oxide (e.g., an iron oxide having a ferrihydrite or lepidocrocite structure), manganese oxide, and the like. The metal used here contains silicon and phosphorus. The metal oxide may be in the shape of a microtube, a nanotube, a hollow string, a capsule, a string-and-sphere agglomerate, a string, a rod, or the like. The size of the metal oxide is preferably as follows. Microtubular metal oxide: diameter of 0.3 to 4 gm, length of 5 to 200 μm; nanotubular metal oxide: diameter of 300 to 450 nm, length of 5 to 200 μm; hollow string-shaped metal oxide: length of 3 to 10 μm; capsule-shaped metal oxide: major axis of 0.5 to 7 μm, minor axis of 0.5 to 3 μm; thread-shaped metal oxide: length of 0.5 to 5 μm; and rod-shaped metal oxide: length of 5 to 30 μm.

With regard to the method for culturing the microorganism, the media and culture methods described in the section “Culture medium for culturing bacterium capable of producing metal oxide” can be used.

One example of a method for collecting a metal oxide from a culture fluid is to replace the supernatant of the medium with distilled water several times to remove the medium components, and subject the metal oxide to air drying to collect them.

### Iron Oxide

The iron oxide of the present invention has a ferrihydrite or lepidocrocite structure that is an aggregate of ferrihydrite nanoparticles or lepidocrocite nanoparticles, and has a fibrous or scaly surface.

The surface refers to the outer surface of the tube. The term “fibrous” refers to the state of a surface where thread-like materials are tangled with each other in a complex manner. The term “scaly” refers to a surface that is covered with scaly substances.

The iron oxide may be in the shape of a microtube, a nanotube, a hollow string, a capsule, a string-and-sphere agglomerate, a string, a rod, or the like. The size of the metal oxide is preferably as follows. Microtubular metal oxide: diameter of 0.3 to 4 μm, length of 5 to 200 μm; nanotubular metal oxide: diameter of 300 to 450 nm, length of 5 to 200 μm; hollow string-shaped metal oxide: length of 3 to 10 μm; capsule-shaped metal oxide: major axis of 0.5 to 7 μm, minor axis of 0.5 to 3 μm; thread-shaped metal oxide: length of 0.5 to 5 μm; and rod-shaped metal oxide: length of 5 to 30 μm.

The components of the iron oxide of the present invention include, for example, Fe, O, Si, and P. The iron oxide typically further includes a carbon atom and a hydrogen atom. It is usually preferable that the element ratio of iron, silicon, and phosphorus is approximately 66-87:2-27:1-32 by atomic % (at %). The primary particle diameter of the ferrihydrite nanoparticles of the iron oxide of the present invention is preferably about 3 to 5 nm, and the primary particle diameter of the lepidocrocite nanoparticles thereof is preferably about 30 to 50 nm.

The iron oxide can be produced by the method explained in the section “Method for producing a metal oxide” above.

### Magnetic Iron Oxide

The iron oxide may be heat-treated to impart magnetism to obtain a magnetic iron oxide. The heat-treatment conditions are not particularly limited, insofar as the iron atom contained in the iron oxide is reduced and oxidized to a magnetic iron oxide (for example, Fe₃O₄ and γ-Fe₂O₃). The heat treatment of the present invention includes heating accompanied by oxidation, heating accompanied by reduction, and heating not accompanied by oxidation or reduction. The heat treatment may be carried out, for example, by an oxidation method comprising heating at 700 to 900° C. in the presence of an oxygen gas (for example, atmospheric air), a hydrogen reduction method comprising heating at about 400 to 650° C. in the presence of hydrogen gas, or a method of mixing a starting material of iron oxide with an aqueous alkali solution containing Fe²⁺ ion prepared by replacement with N² gas and heating the resulting mixture under reflux (see, for example, “S. A. Kahani and M. Jafari, J. Magn. Magn. Mater., 321 (2009) 1951-1954”, etc.).

A preferable method (heat treatment) for producing the magnetic iron oxide is, for example, a method comprising the following steps (1) and (2):

(1) heating the iron oxide; and
(2) reducing the iron oxide obtained in Step (1) by heating in the presence of hydrogen gas.
The heat treatment comprising the above Steps (1) and (2) produces a magnetic iron oxide mainly containing Fe₃O₄.

Another example of a preferable method (heat treatment) for producing the magnetic iron oxide is a method comprising the following Step (3) in addition to the heat treatment comprising the above Steps (1) and (2):

(3) heating the magnetic iron oxide obtained in Step (2) in the presence of oxygen gas (an oxidation-annealing step).

The heat treatment comprising the above Steps (1) to (3) produces a magnetic iron oxide mainly containing γ-Fe₂O₃.

Both in the method comprising Steps (1) and (2), and the method comprising Steps (1) to (3), the iron oxide of the present invention can be made magnetic without performing Step (1).

### EXAMPLES

Hereinafter, the present invention is described in detail with reference to Examples. However, the present invention is not limited to these Examples.

### Example 1

### 1. Isolation of Microorganism of the Present Invention

(1) Isolation of OUMS1 Strain from Water Purification Plant in Joyo City, Kyoto

Water was collected from groundwater sediment contained in an iron bacteria tank in the Joyo City Cultural Center in Joyo City, Kyoto, and placed in a container. A small amount thereof (e.g., 0.5 to 1 g) was introduced into a GP liquid medium (containing 0.076 g of disodium hydrogenphosphate dodecahydrate, 0.02 g of potassium dihydrogenphosphate dihydrate, 2.383 g of HEPES, and 0.01 mM of iron sulfate, per liter of sterile groundwater, whose pH was adjusted to 7.0 with an aqueous sodium hydroxide solution) containing an iron fragment (purity: 99.9%, about 5 mm square), and sufficiently suspended. Thereafter, the resulting product was cultured at 20° C. for 10 days in a shaking incubator (70 rpm). A portion of the sediment that increased during the culture was collected, transferred to a flask containing a fresh GP liquid medium containing an iron fragment, and subjected to shaking culture for another 10 days under the same conditions. This process was repeated once again. A small amount of the liquid in the flask was collected and diluted with a GP liquid medium to 10⁻² to 10⁻⁶. Each diluted solution was separately added dropwise to a respective GP agar plate medium in a sterile Petri dish, and spread-plated onto each of the media with a sterile glass rod. When the media were cultured at 20° C. for 7 to 10 days in an incubator, the proliferation of the target bacteria and the formation of a sheath-shaped oxide were observed.

After the completion of the culture, the obtained single colony (strain) was individually picked up with a sterilized toothpick, inoculated into newly prepared GP agar plate media, and cultured at 20° C. for 10 days. Colonies then appeared on the media. Among these colonies, an irregularly shaped colony of a light-yellowish brown color was identified. Observation with a low-power optical microscope confirmed that the majority of the moiety of a light-yellowish brown color was in the sheath structure. The isolated strain having such properties was designated as an OUMS1 strain.

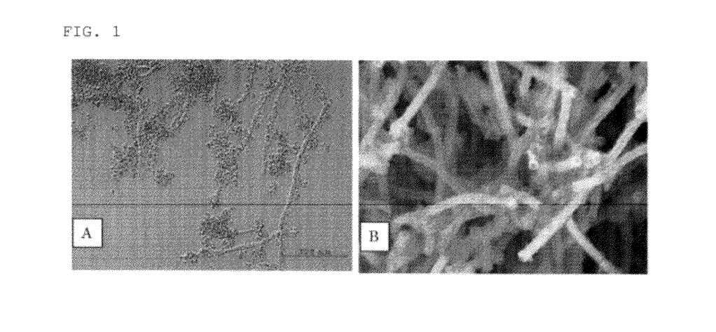

A portion of the identified OUMS1 strain colony was scraped, transferred to a flask containing a newly prepared GP liquid medium, and cultured at 20° C. for 10 days in a shaking incubator (70 rpm). Thereafter, the increased suspended material was placed on a slide glass, and observed with an optical microscope and a scanning electron microscope. The formation of a sheath-shaped oxide was confirmed (FIGS. 1-A and 1-B).

(2) Identification of OUMS1 Strain Isolated from Water Purification Plant in Joyo City, Kyoto

The OUMS1 strain was cultured on a GP agar plate at 23° C. for 10 days. 1 mL of a TE buffer (10 mM Tris/1 mM EDTA) was added to the plate, and the cells were scraped with a cell scraper (produced by TRP) and collected into an Eppendorf tube. Thereafter, the cells were collected by centrifugation at 5,000 g for 10 min. The genomic DNA was extracted by the CTAB method, and the 16S rDNA region was amplified by PCR with the following primers.

5′-AGA GTT TGA TCM TGG CTC AG-3′
5′-GGY TAC CTT GTT ACG ACT T-3′

The amplified fragments were TA-cloned using a TA PCR cloning kit (produced by BioDynamics Laboratory Inc.), and DNA sequencing was performed by the dideoxy method (Sanger method). The obtained DNA sequence was equal to the nucleotide sequence of SEQ ID NO: 1. A homology search was performed for the nucleotide sequence of 16S ribosomal DNA using BLAST of the DDBJ.

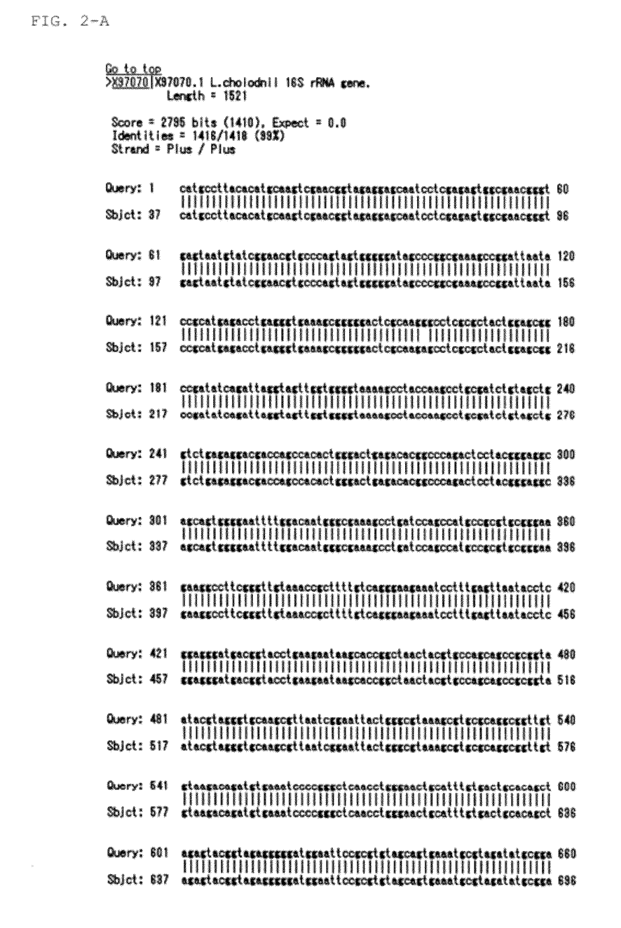

FIGS. 2-A and 2-B show the results of the homology search. The results showed 99% homology with the 16S ribosomal DNA nucleotide sequence (Non-patent Literature 4) of a known iron-oxidizing bacteria Leptothrix cholodnii SP-6 strain (Non-patent Literature 3).

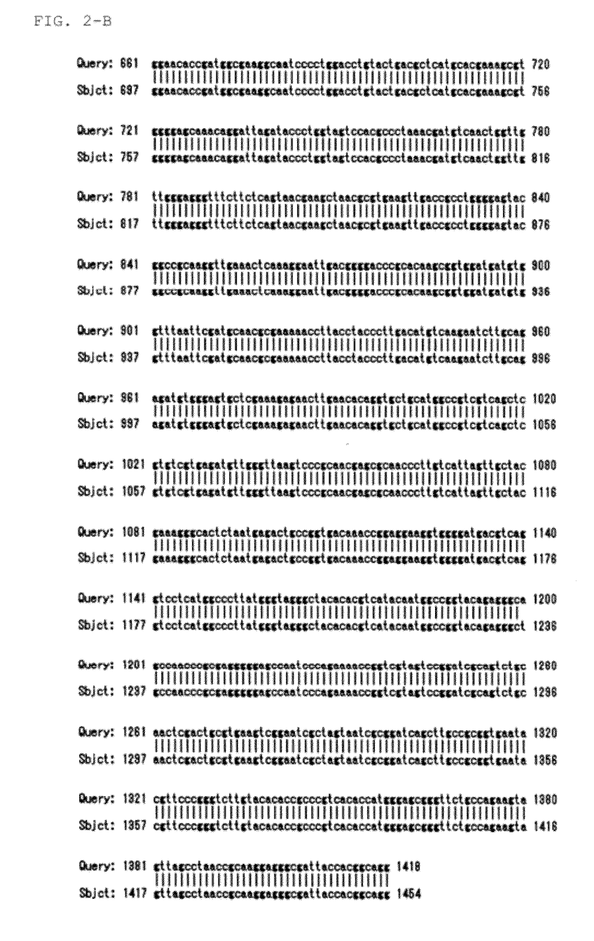

The OUMS1 strain was cultured at 20° C. for 4 days in an MSVP (see Non-patent Literature 2) liquid medium, and the proliferated bacterial cells were collected. Then, the genomic DNA was extracted by the CTAB method, and genomic DNA analysis was performed in accordance with the random amplified polymorphic DNA (RAPD) method, so as to make a comparison with the genomic DNA of a known iron-oxidizing bacteria Leptothrix cholodnii SP-6 strain. FIG. 3 shows the genomic DNA electrophoretic patterns of the OUMS1 strain and a known iron-oxidizing bacteria Leptothrix cholodnii SP-6 strain.

As shown in FIG. 3, in all six types of primers used, the OUMS1 genomic DNA electrophoretic patterns were different from those of known SP-6 in terms of the length and the number of the amplified fragments. This clarifies that the OUMS1 strain differs from SP-6.

A portion of the OUMS1 strain colonies was scraped, transferred to a flask containing an MSVP liquid medium (Non-patent Literature 3) containing manganese sulfate in place of iron sulfate, and cultured at 20° C. for 10 days in a shaking incubator (70 rpm). Thereafter, the increased suspended material was placed on a slide glass and observed with an optical microscope. The formation of a sheath-shaped oxide was confirmed.

The OUMS1 strain was the same as a known iron-oxidizing bacteria Leptothrix cholodnii SP-6 strain in terms of the shape of the culture colonies, sheath-shaped oxide formation capability, and manganese oxidation capability. Further, because the results of the homology search for the 16S ribosomal DNA nucleotide sequence confirmed that the OUMS1 strain showed 99% homology with a known iron-oxidizing bacteria Leptothrix cholodnii SP-6 strain, the OUMS1 strain was identified as known iron-oxidizing bacteria Leptothrix cholodnii. In addition, because a comparison of the genomic DNA electrophoretic patterns by the RAPD method confirmed that the OUMS1 strain differs from a known iron-oxidizing bacteria Leptothrix cholodnii SP-6 strain, the OUMS1 strain was designated as Leptothrix cholodnii OUMS1 strain (NITE BP-860).

(3) Long-Term Storage of Leptothrix cholodnii OUMS1 Strain

Leptothrix cholodnii OUMS1 strain was cultured in a GP or MSVP liquid medium for 4 days. 1 mL thereof was suspended with 0.5 mL of 50% by volume sterile glycerol liquid. The suspension was cryopreserved at −80° C., and freeze-thawed after 14 months. Thereafter, the resulting product was transferred to a manganese sulfate-containing MSVP liquid medium, and cultured at 20° C. in a shaking incubator (70 rpm). The proliferation capability and sheath-shaped oxide formation capability were confirmed.

By performing cryopreservation by the method described above, it was confirmed that the OUMS1 strain's proliferation capability and sheath-shaped oxide formation capability were maintained for at least 14 months.

### 2. Optimal Culture Condition for Promoting Proliferation of OUMS1 Strain and Facilitating Formation of Sheath-Shaped Oxide

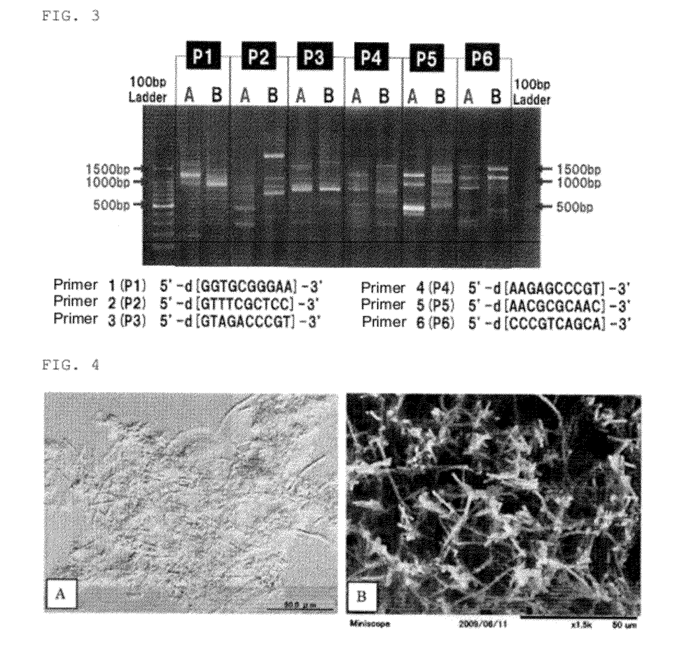

OUMS1 strain was introduced into an SIGP liquid medium (containing 1 g of glucose, 1 g of peptone, 0.2 g of sodium metasilicate nonahydrate, 0.044 g of calcium chloride dihydrate, 0.041 g of magnesium sulfate heptahydrate, 0.076 g of disodium hydrogenphosphate dodecahydrate, 0.02 g of potassium dihydrogenphosphate dihydrate, 2.383 g of HEPES, and 0.05 mM of iron sulfate, per liter of sterile distilled water, whose pH was adjusted to 7.0 with an aqueous sodium hydroxide solution) containing an iron fragment (purity: 99.9%, about 5 mm square), and sufficiently suspended. Thereafter, the resulting product was cultured at 20° C. for 21 days in a shaking incubator (70 rpm). After the completion of the culture, the surface of the iron fragment and the increased suspended material were observed with an optical microscope and a scanning electron microscope. The formation of a sheath-shaped oxide was confirmed (FIGS. 4-A and 4-B).

### 3. Properties of Iron Oxide Formed by OUMS1

The crystal structure of the iron oxide formed by the OUMS1 strain was measured using X-ray diffraction (XRD), its composition was analyzed by energy-dispersive X-ray (EDX) analysis, and the microstructural observation was evaluated with a scanning electron microscope (SEM) and a transmission electron microscope (TEM).

FIGS. 5-A-1 to 5-A-14 and 5-B-1 and 5-B-2 show SEM images of the iron oxide formed by the OUMS1 strain. It was clear that almost all of the visible structures had a tubular (microtubular) shape on the order of microns. The outer diameter of the structure was about 1.6 to 3.7 μm, and the internal diameter was about 0.5 to 0.8 μm. The surface shape of the iron oxide formed by the OUMS1 strain can be roughly classified into three shapes. Specifically, a surface shape such that fibrous particles (fiber width: about 100 to 200 nm) are sparsely tangled, as shown in FIGS. 5-A-1 to 5-A-6; a surface shape such that fibrous particles (fiber width: about 100 to 300 nm) are densely tangled, as shown in FIGS. 5-A-7 to 5-A-11; and a surface shape comprising scaly particles, as shown in FIGS. 5-A-12 to 5-A-14. In addition to these, an agglomerate, as shown in FIG. 5-B-1; and a rod-shaped iron oxide having a thickness of about 1 μm, as shown in FIG. 5-B-2, were also observed.

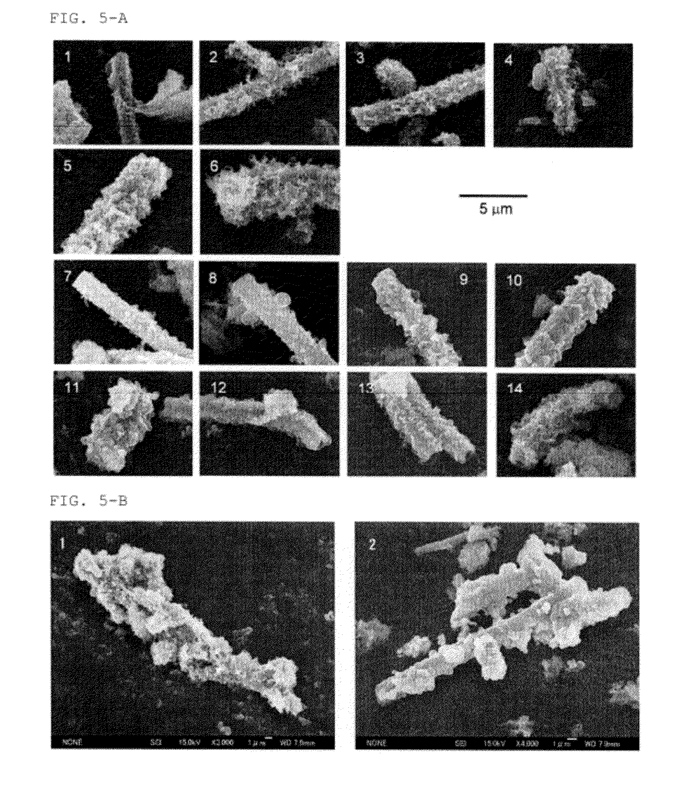

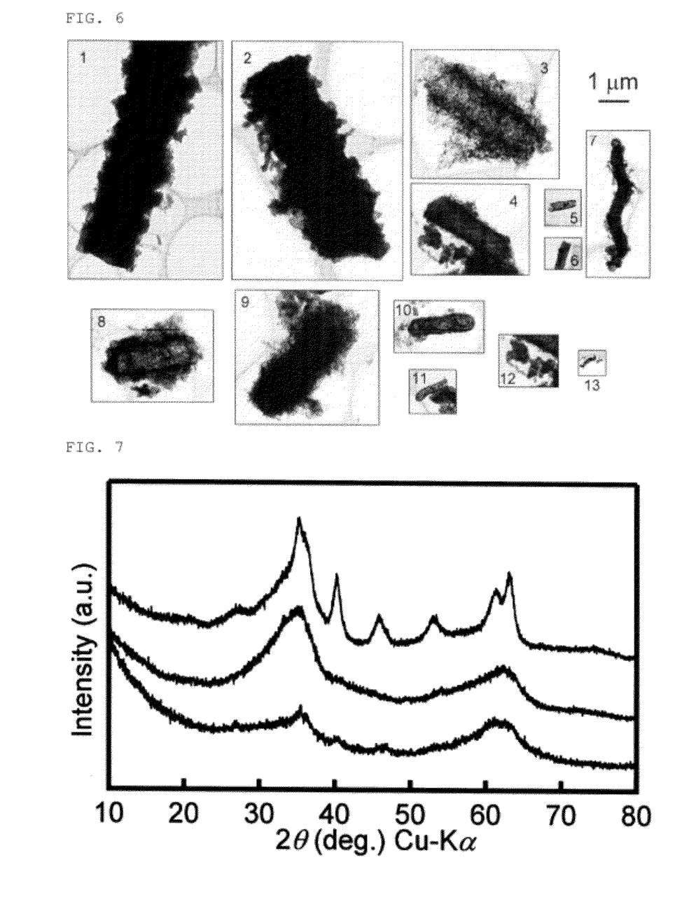

FIGS. 6-1 to 6-13 show TEM images of the iron oxide formed by the OUMS1. In addition to the shapes shown in FIGS. 6-1 to 6-4, which are similar to the microtubular shapes observed in the SEM images above, the following shapes were confirmed: a nanotubular shape having an outer diameter of about 350 to 400 nm, as shown in FIGS. 6-5 and 6-6; a hollow string-like shape having an outer diameter of about 500 nm and an internal diameter of about 180 nm, as shown in FIG. 6-7; a capsule shape having a major axis of about 1.5 to 5 μm and a minor axis of about 0.78 to 2.0 μm, as shown in FIGS. 6-8 to 6-10; a tubular shape whose one end is closed, having an outer diameter of about 350 nm and an internal diameter of about 230 nm, as shown in FIGS. 6-11; a string-and-sphere agglomerate, as shown in FIG. 6-12; and a string-like iron oxide, as shown in FIG. 6-13. These results clarified that the OUMS1 formed an iron oxide having various shapes, such as a nanotubular shape; a hollow string shape; a capsule shape; a string-and-sphere agglomerate; and a string-like shape, in addition to a microtube-shaped iron oxide.

As a result of the composition analysis by EDX, it became clear that the constituent components of the iron oxide formed by the OUMS1 were Fe, O, Si, and P. Table 1 shows the average values and the standard deviations of the results of the analysis performed for 24 points. The composition excluding oxygen was Fe:Si:P=79.3:8.8:11.9. This iron oxide also contains a carbon atom and a hydrogen atom.

TABLE 1
Analytical Points: 24

| | |
|---|---|
| Average | Standard deviation |

| Element | wt % | at % | wt % | at % |
|---|---|---|---|---|
| Si K | 4.9 | 8.8 | 1.5 | 2.8 |
| P K | 7.4 | 11.9 | 5.6 | 8.6 |
| Fe K | 87.7 | 79.3 | 4.4 | 6.2 |

FIG. 7 shows an XRD pattern of the iron oxide formed by the OUMS1 strain (lowest), and, as comparison samples, XRD patterns of 2-line ferrihydrite (2^{nd} from the lowest) and 6-line ferrihydrite (3^{rd} from the lowest). The iron oxide formed by the OUMS1 strain shows peaks that appear to be a combination of the peaks of 2-line ferrihydrite and 6-line ferrihydrite. These results clarified that the iron oxide formed by the OUMS1 was ferrihydrite.

FIG. 8 shows a high-resolution transmission electron microscope (HRTEM) image of a typical microtubular iron oxide formed by the OUMS1. This clarified that the iron oxide formed by the OUMS1 had a primary particle diameter of about 3 to 5 nm. Further, clear cross stripes were observed in the primary particles. This clarified that the iron oxide formed by the OUMS1 was a microcrystal aggregate.

The results of XRD measurement and HRTEM observation clarified that the iron oxide formed by the OUMS1 was an aggregate of ferrihydrite fine particles, the primary particle diameter thereof being about 3 to 5 nm.

### Example 2

### 1. Optimal Culture Condition for Promoting Proliferation of OUMS1 Strain and Facilitating Formation of Sheath-Shaped Lepidocrocite Oxide

Using the OUMS1 isolated in Example 1, lepidocrocite was prepared under the following culture conditions.

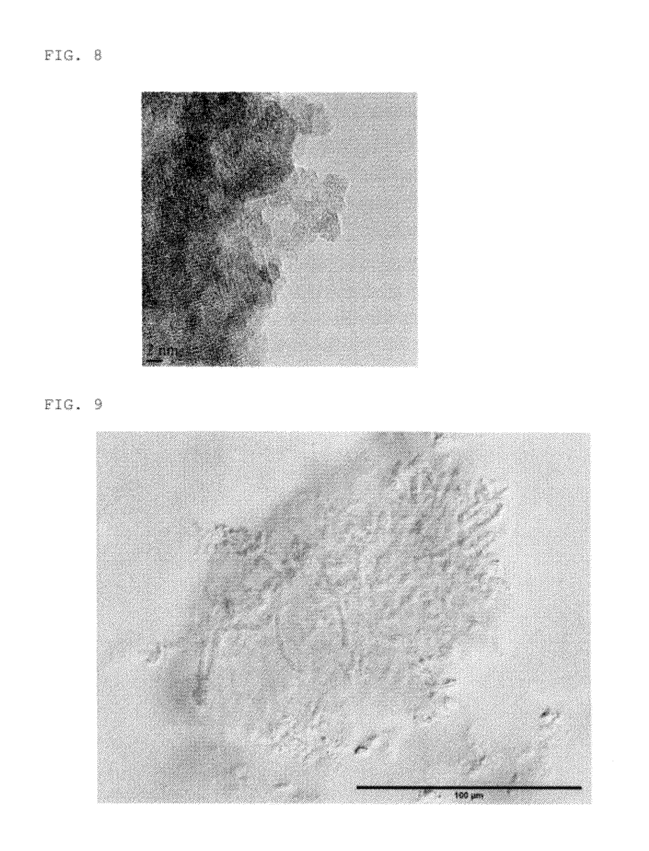

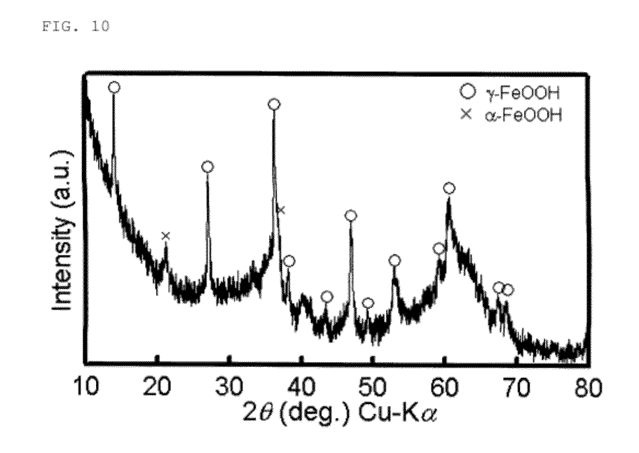

OUMS1 strain was introduced into an SIGP liquid medium (containing 1 g of glucose, 1 g of peptone, 0.2 g of sodium metasilicate nonahydrate, 0.044 g of calcium chloride dihydrate, 0.041 g of magnesium sulfate heptahydrate, 0.076 g of disodium hydrogenphosphate dodecahydrate, 0.02 g of potassium dihydrogenphosphate dihydrate, 2.383 g of HEPES, and 0.05 mM of iron sulfate, per liter of sterile distilled water, whose pH was adjusted to 7.0 with an aqueous sodium hydroxide solution) containing three pieces of iron fragments (purity: 99.9%, about 1 cm square), and sufficiently suspended. Thereafter, the resulting product was cultured at 20° C. for 14 days in a shaking incubator (70 rpm). After the completion of the culture, the surfaces of the iron fragments and the increased suspended material were observed with an optical microscope and a scanning electron microscope. The formation of a sheath-shaped oxide was confirmed (FIG. 9). Collected sediment was washed with about ten times the amount of distilled water, and then dried under reduced pressure. The XRD measurement of the dry powder revealed that the resulting sheath-shaped oxide was lepidocrocite (FIG. 10). Slight peaks attributable to goethite (α-FeOOH) were also confirmed. It revealed that the crystallite size (the minimum crystallite size in the direction perpendicular to the (200) plane) calculated based on the half-widths of reflection of (200) planes in an XRD pattern was 30 nm.

### Devices Used for Analysis

Optical microscope: Olympus, BX-51 (FIGS. 1-A, 4-A, and 9)
X-ray diffraction (XRD) measurement: Rigaku Corporation, RINT-2000 (FIGS. 7 and 10)
Scanning electron microscope (SEM): Hitachi High-Technologies Corporation, Miniscope TM-1000 (FIGS. 1-B and 4-B)
Scanning electron microscope (SEM): JEOL Ltd., JSM-6700F (FIGS. 5-A and 5-B)
Energy Dispersive X-Ray (EDX) analysis: JEOL Ltd., JED-2200F (Table 1)
Transmission electron microscope (TEM): JEOL Ltd., JEM-2100F (FIGS. 6 and 8)

1-13. (canceled)

14. A microorganism that belongs to the genus Leptothrix,
the microorganism being capable of producing an iron oxide having a structure of ferrihydrite or lepidocrocite, the iron oxide being an aggregate of ferrihydrite nanoparticles or lepidocrocite nanoparticles.

15. The microorganism according to claim 14, wherein the iron oxide comprises phosphorus and silicon.

16. The microorganism according to claim 14, which comprises 16S rDNA consisting of the nucleotide sequence of SEQ ID NO: 1

17. The microorganism according to claim 14, which is Leptothrix cholodnii OUMS1 (NITE BP-860).

18. A bacterium that is capable of producing an iron oxide having a structure of ferrihydrite or lepidocrocite, the iron oxide being an aggregate of ferrihydrite nanoparticles or lepidocrocite nanoparticles.

19. The bacterium that is capable of producing an iron oxide according to claim 18, wherein the iron oxide contains phosphorus and silicon.

20. A method for screening the microorganism of claim 14 or the bacterium that is capable of producing an iron oxide of claim 18 comprising culturing the microorganism or bacterium using a culture medium comprising an inorganic phosphorus compound and an iron compound added to natural ground water.

21. A method for culturing the microorganism of claim 14 or the bacterium that is capable of producing an iron oxide of claim 18, the method comprising a step of using a culture medium comprising as medium components a carbon source, a nitrogen source, silicon, sodium, calcium, magnesium, potassium, inorganic phosphate, and iron.

22. A method for producing a metal oxide comprising:
culturing the microorganism of claim 14 or the bacterium that is capable of producing an iron oxide of claim 18; and
collecting the metal oxide from a culture fluid.

23. The method according to claim 22, wherein the metal oxide is in the shape of a microtube, a nanotube, a hollow string, a capsule, a string-like and sphere-like agglomerate, a string, or a rod.

24. An iron oxide having a ferrihydrite or lepidocrocite structure, being an aggregate of ferrihydrite nanoparticles or lepidocrocite nanoparticles, and having a fibrous or scaly surface.


SEQUENCE LISTING (9 sequences)

SEQ ID NO: 1 - DNA, 1418 bp, Leptothrix cholodnii
catgccttacacatgcaagtcgaacggtagaggagcaatcctcgagagtggcgaacgggtgagtaatgtatcggaacgtgcccagtagtgggggatagcccggcgaaagccggattaataccgcatgagacctgagggtgaaagcgggggactcgcaagggcctcgcgctactggagcggccgatatcagattaggtagttggtggggtaaaagcctaccaagcctgcgatctgtagctggtctgagaggacgaccagccacactgggactgagacacggcccagactcctacgggaggcagcagtggggaattttggacaatgggcgaaagcctgatccagccatgccgcgtgcgggaagaaggccttcgggttgtaaaccgcttttgtcagggaagaaatcctttgagttaatacctcggagggatgacggtacctgaagaataagcaccggctaactacgtgccagcagccgcggtaatacgtagggtgcaagcgttaatcggaattactgggcgtaaagcgtgcgcaggcggttgtgtaagacagatgtgaaatccccgggctcaacctgggaactgcatttgtgactgcacagctagagtacggtagagggggatggaattccgcgtgtagcagtgaaatgcgtagatatgcggaggaacaccgatggcgaaggcaatcccctggacctgtactgacgctcatgcacgaaagcgtggggagcaaacaggattagataccctggtagtccacgccctaaacgatgtcaactggttgttgggagggtttcttctcagtaacgaagctaacgcgtgaagttgaccgcctggggagtacggccgcaaggttgaaactcaaaggaattgacggggacccgcacaagcggtggatgatgtggtttaattcgatgcaacgcgaaaaaccttacctacccttgacatgtcaagaatcttgcagagatgtgggagtgctcgaaagagaacttgaacacaggtgctgcatggccgtcgtcagctcgtgtcgtgagatgttgggttaagtcccgcaacgagcgcaacccttgtcattagttgctacgaaagggcactctaatgagactgccggtgacaaaccggaggaaggtggggatgacgtcaggtcctcatggcccttatgggtagggctacacacgtcatacaatggccggtacagagggcagccaacccgcgagggggagccaatcccagaaaaccggtcgtagtccggatcgcagtctgcaactcgactgcgtgaagtcggaatcgctagtaatcgcggatcagcttgccgcggtgaatacgttcccgggtcttgtacacaccgcccgtcacaccatgggagcgggttctgccagaagtagttagcctaaccgcaaggagggcgattaccacggcagg

SEQ ID NO: 2 - DNA, 20 bp, Artificial Sequence
  primer
agagtttgatcmtggctcag

SEQ ID NO: 3 - DNA, 19 bp, Artificial Sequence
  primer
ggytaccttgttacgactt

SEQ ID NO: 4 - DNA, 10 bp, Artificial Sequence
  primer
ggtgcgggaa

SEQ ID NO: 5 - DNA, 10 bp, Artificial Sequence
  primer
gtttcgctcc

SEQ ID NO: 6 - DNA, 10 bp, Artificial Sequence
  primer
gtagacccgt

SEQ ID NO: 7 - DNA, 10 bp, Artificial Sequence
  primer
aagagcccgt

SEQ ID NO: 8 - DNA, 10 bp, Artificial Sequence
  primer
aacgcgcaac

SEQ ID NO: 9 - DNA, 10 bp, Artificial Sequence
  primer
cccgtcagca